(19) United States
(12) Patent Application Publication
(10) Pub. No.: US 2005/0048017 A1
(43) Pub. Date: Mar. 3, 2005

(54) Use of surfactants with a ratio of hemolysis value to denaturation index of greater than or equal to 1 for obtaining or increasing the selectivity of cleansing preparations

(76) Inventors: Manfred Strassner, Hamburg (DE); Stephan Ruppert, Hamburg (DE); Andreas Schepky, Hamburg (DE); Jens-Peter Vietzke, Hamburg (DE); Harald Albrecht, Hamburg (DE); Urte Koop, Hamburg (DE); Ralph Schimpf, Bonningstedt (DE); Joachim Ennen, Hamburg (DE); Claudius Rapp, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/490,890

(22) PCT Filed: Aug. 23, 2002
(86) PCT No.: PCT/EP02/09420

(30) Foreign Application Priority Data
Sep. 25, 2001 (DE) 10147049.5

(51) Int. Cl. A61K 7/06; A61K 7/11

(52) U.S. Cl. 424/70.13; 424/70.16

(57) ABSTRACT

The invention relates to the use of one or more surface-active substances selected from the group consisting of surfactants, which have a ratio of hemolysis value to denaturation index of greater than or equal to 1, for obtaining or increasing the selectivity of cosmetic or dermatological cleansing preparations.

The present invention relates to the use of surfactants with a ratio of hemolysis value to denaturation index of greater than or equal to 1 for obtaining or increasing the selectivity of cosmetic or dermatological cleansing preparations.

Even cleansing of the skin using a simple waterbath—without the addition of surfactants—initially causes the horny layer of the skin to swell. The degree of this swelling depends inter alia on the bathing time and temperature. At the same time, water-soluble substances are washed off or out, such as, for example, water-soluble constituents of dirt, but also substances endogenous to the skin which are responsible for the water-binding capacity of the horny layer. In addition, as a result of surface-active substances which are endogenous to the skin, fats in the skin (surface and barrier lipids) are also dissolved and washed out to a certain degree.

While the combined removal of surface dirt and surface lipids which primarily originate from the sebaceous glands and are decisively responsible for the appearance of greasy skin is entirely desirable in cosmetic terms, the washing-out of barrier lipids can impair the function of the skin barrier, which is associated with a loss of moisture from the skin. This is accompanied by a sporadic increase in the transepidermal water loss (TEWL) and a sporadic reduction in skin moisture. Primarily for products for the cleansing of sensitive facial skin and here particularly in the case of products for greasy skin, as selective as possible a removal of surface dirt, make-up and skin sebum is particularly important for the consumer. On the other hand, the facial skin in particular requires particularly gentle cleansing.

In healthy skin, the disturbances caused by washing are generally limited in terms of time since the protective mechanisms of the skin are able to readily compensate for such slight disturbances to the upper layers of the skin. However, even in the case of nonpathological deviations from the norm, e.g. as a result of wear damage or irritations caused by the environment, photodamage, aging skin etc., the protective mechanism of the surface of the skin is impaired. In some instances, it is then no longer able by itself to fulfill its function and has to be regenerated by external measures. There has therefore been no lack of attempts to find suitable cleansing preparations for better and more rapid regeneration of the skin which help the skin to retain its natural balance.

Known compositions for the cleansing and simultaneous care of the skin are, for example, oil-containing cleansing preparations or cleansing preparations comprising various refatting agents or skin moisturizers, which are intended to refat or remoisturize the skin at the same time as cleansing it. For this purpose, the prior art recognizes, for example, oilbath preparations of various types and also shower oils, cleansing creams and the like.

The main disadvantage of such preparations is that some of the barrier lipids are initially removed and the skin is then refatted with the help of the added oil components. In addition, such cleansing preparations are washed off following application, meaning that only small amounts of the additives used remain on the skin.

It was therefore an object of the present invention to provide cosmetic or dermatological preparations which significantly improve the condition of the skin, in particular reduce the roughness of the skin, and which are characterized by a selectivity of the cleansing performance.

It was surprising and could in no way have been foreseen by the person skilled in the art that
the use of one or more interface-active substances chosen from the group of surfactants which have a ratio L/D of hemolysis value (L) and denaturation index (D) of greater than or equal to 1 for obtaining or enhancing the selectivity of cosmetic or dermatological cleaning preparations
   would overcome the disadvantages of the prior art.

Selective cleansing preparations within the meaning of the present invention wash significantly more surface or sebum lipids out than barrier lipids. Thus, if the ratio of washed out surface or sebum lipids to washed out barrier lipids is defined, then this is significantly greater than 1 for preparations comprising surfactants used according to the invention with a ratio L/D of greater than or equal to 1, preferably greater than or equal to 3, with particular preference greater than or equal to 5. The increase in the selectivity of a cleansing preparation can be determined according to the invention, for example, relative to water. According to the present invention, it is possible to formulate products which remove dirt and excess sebum with high selectivity and in so doing conserve the lipids endogenous to the skin which are essential for preventing the skin from drying out.

The ratio L/D represents the relationship between hemolysis value (L) and denaturation index (D) and is determined by means of the standard RBC test.

The standard RBC test serves for estimating in vivo ocular mucosa irritation potentials of surfactants and surfactant products. The method is based on the fact that surfactants interact strongly with cell membranes and proteins. Both effects are determined photometrically by analyzing the natural blood dye oxyhemoglobin (HbO₂). In contrast to other cell-based systems the RBC test is capable of differentiating between damage to the cell membrane (hemolysis) and protein denaturation (denaturation index).

Fresh samples of calf blood are obtained direct from the abattoir. The red blood cells are repeatedly washed and centrifuged in order to separate off the white blood cells and to remove all residues of plasma.

1. Hemolysis:

A defined aliquot of isolated calf erythrocytes is incubated with shaking at RT for 10 minutes with a series of increasing concentration of the specimens containing the washing-active substances under analysis (stock solution for raw materials containing 0.1% by weight of the washing-active substance in PBS). PBS is a customary standard phosphate buffer (pH 7.4) whose composition is as follows:

| | | |
|---|---|---|
| Na₂HPO * 2 H₂O | 3.95 | g |
| KH₂PO₄ | 0.76 | g |
| NaCl | 7.20 | g |
| Glucose * 1 H₂O | 1.80 | g |
| Aqua dest. or highly purified water | ad 1000 | ml |

The incubation period is ended by rapid high-speed centrifuging. After centrifuging, the supernatants recovered are analyzed photometrically for the amount of liberated hemoglobin (HbO2) therein at 530 nm. The relative degree of hemolysis, based on 100% hemolysis, is calculated from this, and the L value [μl/ml] (generally also referred to as the H50 value) is determined as a parameter from the concentration/response curve. This L value indicates the concentration of the test sample at which 50% of the hemoglobin is released.

2. HbO2 Denaturation Index:

A defined aliquot of isolated calf erythrocytes is incubated with shaking at room temperature for 10 minutes with a fixed concentration of the test sample (in the case of surfactants: 0.1% of active substance in PBS) and then centrifuged rapidly. The change in the spectral absorption at 575 nm and 540 nm is measured in comparison to the native HbO2. From the ratio of the absorption values to one another the denaturation index D [%] is calculated. Na lauryl sulfate (0.1% active substance) serves as 100% standard.

L/D Ratio:

The ratio represents the relationship between the parameters of hemolysis (L) and denaturation index (D) and is used for classifying and characterizing the test samples.

The method is also recorded in the INVITTOX database for the recording of alternatives to animal experimentation, under protocol No. 37, and is further described in the following references:
1. Kondo, T. (1976) Mechanisms of haemolysis by surface active agents. Adv. Colloid & Interface Sci., 6, 139-172 Kondo, T. & Tomizawa, M. (1968) Haemolysis by nonionic surface-active agents. J. Pharm. Sci., 57, 1246-1248.
2. Gloxhuber, Ch. (1974) Toxicological properties of surfactants. Arch. Toxicol., 32, 245-270.
3. Pape, W. J. W., Pfannenbecker, U. & Hoppe U. (1987) Validation of the red blood cell test as an in vitro assay for the rapid screening of irritation potential of surfactants. Molecular Toxicology, 1, 525-536.
4. Pape, W. & Hoppe, U. (1988) Second World Surfactants Congress, Paris. Evaluation of acute irritation potentials of tensides using the in vitro alternative red blood cell test system. Proceedings, IV, 414-428.
5. Pape, W. J. W. (1990) In vitro methods for the assessment of local effects of cosmetics on skin and mucous membranes. Presented at: In-cosmetics 1990, Birmingham, UK.
6. Pape, W. J. W. & Hoppe, U. (1991) Standardisation on an in vitro red blood cell test for evaluating the acute cytotoxic potential of tensides. Arzneimittel-Forschung/Drug Research, 40(I), 4, 498-502.
7. Pape, W. J. W. & Hoppe, U. (1991) In vitro methods for the assessment of primary local effects of topically applied preparations. Skin Pharmacology (in press).

The surfactant or surfactants for use in accordance with the invention are advantageously chosen from the group of surfactants having a ratio L/D of hemolysis value (L) to denaturation index (D) of greater than or equal to 3, with particular preference greater than or equal to 5.

With particular advantage the surfactant or surfactants for use in accordance with the invention are chosen from the group consisting of sodium N-alkylylglutamates, sodium alkylsarcosinates, sodium alkylsulfosuccinates and sodium salts of ethoxylated and carboxylated triglycerides.

Particular preference for the purposes of the present invention is given to the use of one or more of the following surfactants: sodium cocoylglutamate, sodium lauroyl-sarcosinate, disodium lauryl polyglycol ether sulfosuccinate, sodium PEG-7 olive oil carboxylate.

The total amount of one or more surfactants used according to the invention in the finished cosmetic or dermatological preparations is advantageously chosen from the range from 0.1 to 10.0% by weight, preferably 0.5 to 5.0% by weight, in each case based on the total weight of the preparations.

The cleaning compositions comprising surfactants used according to the invention can have the customary composition and serve for the cosmetic and/or dermatological cleansing of the skin and/or the hair and as a make-up product for decorative cosmetics.

For use, the compositions comprising surfactants used according to the invention are applied to the skin and/or the hair in a sufficient amount in the manner customary for cosmetics.

The cleansing preparations for the purposes of the present invention advantageously comprise one or more washing-active surfactants in the following four groups A to D:

A. Anionic Surfactants

Anionic surfactants to be used advantageously are

acylamino acids (and salts thereof), such as
1. acyl glutamates, for example sodium acyl glutamate, di-TEA-palmitoyl aspartate and sodium caprylic/capric glutamate,
2. acylpeptides, for example palmitoyl-hydrolyzed milk protein, sodium cocoyl-hydrolyzed soya protein and sodium/potassium cocoyl-hydrolyzed collagen,
3. sarcosinates, for example myristoyl sarcosine, TEA-lauroyl sarcosinate, sodium lauroyl sarcosinate and sodium cocoyl sarcosinate,
4. taurates, for example sodium lauroyl taurate and sodium methylcocoyl taurate,
   carboxylic acids and derivatives, such as
1. carboxylic acids, for example lauric acid, aluminum stearate, magnesium alkanolate and zinc undecylenate,
2. ester carboxylic acids, for example calcium stearoyl lactylate, laureth-6 citrate and sodium PEG-4 lauramide carboxylate,
3. ethercarboxylic acids, for example sodium laureth-13 carboxylate and sodium PEG-6 cocamide carboxylate,
   phosphoric esters and salts, such as, for example, DEA-oleth-10 phosphate and dilaureth-4 phosphate,
   sulfonic acids and salts, such as
1. acyl isethionates, e.g. sodium/ammonium cocoyl isethionate,
2. alkylarylsulfonates,
3. alkylsulfonates, for example sodium cocomonoglyceride sulfate, sodium C₁₂₋₂₄ olefinsulfonate, sodium lauryl sulfoacetate and magnesium PEG-3 cocamide sulfate,
4. sulfosuccinates, for example dioctyl sodium sulfosuccinate, disodium laureth sulfosuccinate, disodium lauryl sulfosuccinate and disodium undecyleneamido-MEA sulfosuccinate
   and
   sulfuric esters, such as
1. alkyl ether sulfate, for example sodium, ammonium, magnesium, MIPA, TIPA laureth sulfate, sodium myreth sulfate and sodium C₁₂₋₁₃ pareth sulfate,
2. alkyl sulfates, for example sodium, ammonium and TEA lauryl sulfate.
   B. Cationic Surfactants

Cationic surfactants which can be used advantageously are
1. alkylamines,
2. alkylimidazoles,
3. ethoxylated amines and
4. quaternary surfactants.

Quaternary surfactants contain at least one N atom which is covalently bonded to 4 alkyl or aryl groups. Irrespective of the pH, this leads to a positive charge. Benzalkonium chloride, alkylbetaine, alkylamidopropylbetaine and alkylamidopropyl-hydroxysultaine are advantageous.

C. Amphoteric Surfactants

Amphoteric surfactants which can be used advantageously are
1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,
2. N-alkylamino acids, for example aminopropylalkylglutamide, alkylaminopropionic acid, sodium alkylimidodipropionate and lauroamphocarboxyglycinate.
3. N-alkyl- or N-alkenylbetaines with at least 12 carbon atoms, such as, for example, laurylamidopropylbetaine and oleylamidopropylbetaine.
   D. Nonionic Surfactants

Nonionic surfactants which can be used advantageously are
1. alcohols,
2. alkanolamides, such as cocamides MEA/DEA/MIPA,
3. amine oxides, such as cocoamidopropylamine oxide,
4. esters which are formed by esterification of carboxylic acids with ethylene oxide, glycerol, sorbitan or other alcohols,
5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and coco glycoside.
6. Sucrose esters, sucrose ethers
7. Polyglycerol esters, diglycerol esters, monoglycerol esters
8. Methylglucose esters, esters of hydroxy acids.

The cleansing preparations for the purposes of the present invention particularly advantageously comprise one or more washing-active surfactants according to the invention from the group of surfactants which have an HLB value of more than 25, very particularly those which have an HLB value of more than 35.

It is advantageous for the purposes of the present invention when the content of one or more washing-active surfactants in the cosmetic or dermatological cleaning preparation is chosen from the range from 1.5 to 20% by weight, very particularly advantageously from 2.0 to 15% by weight, in each case based on the total weight of the preparation.

The compositions for the purposes of the present invention further advantageously comprise water and optionally the additives customary in cosmetics, e.g. preservatives, preserving aids, bactericides, perfumes, dyes, pigments which have a coloring effect, moisturizing and/or humectant substances, fillers which improve the feel on the skin, fats, oils, waxes or other customary constituents of a cosmetic or dermatological formulation.

Advantageous preservatives for the purpose of the present invention are, for example, formaldehyde donors (such as, for example, DMDM hydantoin, which is available, for example, under the trade name Glydant™ from Lonza), iodopropyl butylcarbamates (e.g. those available under the trade names Glycacil-L, Glycacil-S from Lonza and/or Dekaben LMB from Jan Dekker), parabens (i.e. p-hydroxybenzoic alkyl esters, such as methyl-, ethyl-, propyl- and/or butylparaben), phenoxyethanol, ethanol, benzoic acid and the like. According to the invention, the preservative system usually also advantageously comprises preserving aids, such as, for example, octoxyglycerol, glycine soya etc.

The water phase of the preparations for the purposes of the present invention can advantageously comprise customary cosmetic auxiliaries, such as, for example, alcohols, in particular those of low carbon number, preferably ethanol and/or isopropanol, diols or polyols of low carbon number, and ethers thereof, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, dihydroxyacetone, and in particular one or more thickeners which can advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination. Moisturizers can also preferably be used.

Moisturizers is the term used to described substances or mixtures of substances which, following application or distribution on the surface of the skin, confer on cosmetic or dermatological preparations the property of reducing the moisture loss by the horny layer (also called transepidermal water loss (TEWL) and/or have a beneficial effect on the hydration of the horny layer.

Advantageous moisturizers for the purposes of the present invention are, for example, glycerol, lactic acid and/or lactates, in particular sodium lactate, butylene glycol, propylene glycol, biosaccharide gum-1, glycine soya, ethylhexyloxyglycerol, pyrrolidonecarboxylic acid and urea. In addition, it is particularly advantageous to use polymeric moisturizers from the group of polysaccharides which are soluble in water and/or swellable in water and/or gellable using water. Particularly advantageous are, for example, hyaluronic acid, chitosan and/or a fucose-rich polysaccharide, which is listed in Chemical Abstracts under the registry number 178463-23-5 and is available, for example, under the name Fucogel® 1000 from SOLABIA S.A.

Particularly advantageous preparations are also obtained when antioxidants are used as additives or active ingredients. According to the invention, the preparations advantageously comprise one or more antioxidants. Favorable, but nevertheless optional, antioxidants which may be used are all antioxidants customary or suitable for cosmetic and/or dermatological applications.

For the purposes of the present invention, water-soluble antioxidants may be used particularly advantageously, such as, for example, vitamins, e.g. ascorbic acid and derivatives thereof.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to choose their respective concentrations from the range from 0.001 to 10% by weight, based on the total weight of the formulation.

If vitamin A or vitamin A derivatives, or carotenes or derivatives thereof are the antioxidant or the antioxidants, it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin and/or the hair against oxidative stress.

Further advantageous active ingredients for the purposes of the present invention are natural active ingredients and/or derivatives thereof, such as, for example, alpha-lipoic acid, phytoene, D-biotin, coenzyme Q10, alpha-glucosylrutin, carnitine, carnosine, natural and/or synthetic isoflavonoids, creatine, taurine and/or β-alanine.

The examples below are intended to illustrate the present invention. The numerical values in the examples are percentages by weight, based on the total weight of the respective preparations.

### EXAMPLES

### Formulation Examples

| | 1 | 2 | 3 | 4 | 5 |
|---|---|---|---|---|---|
| Sodium cocoylglutamate | 0.5 | — | — | 1.0 | 1.0 |
| Sodium | — | 0.5 | — | — | — |
| lauroylsarcosinate | | | | | |
| Disodium lauryl | — | — | 0.5 | — | 0.5 |
| polyglycol ether | | | | | |
| sulfosuccinate | | | | | |
| Sodium PEG-7 olive oil | — | — | — | 1.0 | 0.5 |
| carboxylate | | | | | |
| Decyl glucoside | 0.5 | — | 0.25 | — | — |
| Lauryl glucoside | — | 0.5 | 0.25 | — | — |
| Sodium lauryl ether | 1.5 | — | 1.5 | — | — |
| sulfate | | | | | |
| Sodium myristyl ether | — | 1.5 | — | 1.5 | 1.0 |
| sulfate | | | | | |
| Sodium methyl cocoyl | 0.6 | 0.5 | 0.3 | — | — |
| taurate | | | | | |
| Glycerol | 2.0 | 2.0 | 1.0 | — | — |
| Propylene glycol | — | — | — | 5 | 18 |
| Butylene glycol | — | — | — | 5 | 5 |
| Sodium carbomer | 1.2 | 1.2 | 1.2 | — | — |
| Sodium acrylate/C10-30 | — | — | — | 1.3 | 1.3 |
| alkyl acrylate | | | | | |
| crosspolymer | | | | | |
| Xanthan gum | 0.25 | 0.25 | 0.2 | — | — |
| PEG-40 hydrogenated | — | — | — | 1 | 1 |
| castor oil | | | | | |
| PEG-7 glyceryl cocoate | 0.5 | — | 0.5 | — | — |
| Parabens | 0.2 | 0.2 | 0.15 | 0.15 | 0.15 |
| EDTA | — | — | — | 0.2 | 0.2 |
| Benzophenone-4 | 0.05 | 0.05 | 0.04 | 0.03 | 0.03 |
| Methyldibromoglutaro- | 0.05 | 0.05 | 0.04 | — | — |
| nitrile | | | | | |
| Phenoxyethanol | 0.6 | 0.6 | 0.5 | 0.35 | 0.35 |
| Perfume | qs | qs | qs | qs | qs |
| Water | ad 100 | ad 100 | ad 100 | ad 100 | ad 100 |

1. The use of one or more interface-active substances chosen from the group of surfactants which have a ratio of hemolysis value to denaturation index of greater than or equal to 1, preferably greater than or equal to 3, with particular preference greater than or equal to 5, for obtaining or enhancing the selectivity of cosmetic or dermatological cleaning preparations.

2. The use as claimed in claim 1, characterized in that the surfactants concentration is in the range from 0.1 to 10% by weight, based on the total weight of the formulation.

3. The use as claimed in any of the preceding claims, characterized in that the total concentration of all cleansing-active substances including the surfactants of the invention is in the range from 1.5 to 20% by weight, based on the total weight of the formulation, in particular in the range from 2.0 to 15% by weight.

4. The use as claimed in any of the preceding claims, characterized in that the surfactants are selected from the group of sodium N-alkylylglutamates, sodium alkylsarcosinates, sodium alkylsulfosuccinates and sodium salts of ethoxylated and carboxylated triglycerides, particularly sodium cocoylglutamate, sodium lauroyl-sarcosinate, disodium lauryl polyglycol ether sulfosuccinate, sodium PEG-7 olive oil carboxylate.

5. The use as claimed in any of the preceding claims, characterized in that the cleansing preparations comprise cosurfactants chosen from the group of alkyl ether sulfates and/or alkyltaurates.

6. The use as claimed in any of the preceding claims, characterized in that the cleaning preparations comprise gel formers from the group of polyacrylates and/or acrylate copolymers and/or polysaccharides.

7. The use as claimed in any of the preceding claims, characterized in that the cleansing preparations are used for cleansing the face.

8. The use as claimed in any of the preceding claims, characterized in that the cleansing preparations are preferably used for cleansing greasy and/or unclean skin/scalp.

9. The use as claimed in any of the preceding claims, characterized in that the cleansing preparations comprise further cosmetic auxiliaries, in particular skin moisturizers, oil components, dyes, pigments, preservatives and perfume.

10. The use as claimed in any of the preceding claims, characterized in that the cleansing preparations are suitable for foaming using a pump foamer, preferably a splashproof pump foamer.

11. The use as claimed in any of the preceding claims, characterized in that the cleansing preparations are suitable for cleansing the body.
